(19) United States
(12) Patent Application Publication
(10) Pub. No.: US 2015/0080634 A1
(43) Pub. Date: Mar. 19, 2015

(54) TRACKING EXTERNAL MARKERS TO INTERNAL BODILY STRUCTURES

(71) Applicant: ProNova Solutions, LLC, Knoxville, TN (US)

(72) Inventors: Jonathan Huber, Knoxville, TN (US); Niek Schreuder, Knoxville, TN (US)

(21) Appl. No.: 14/491,303

(22) Filed: Sep. 19, 2014

Related U.S. Application Data
(60) Provisional application No. 61/879,873, filed on Sep. 19, 2013.

(51) Int. Cl. A61N 5/10; A61B 19/00

(52) U.S. Cl. CPC A61N 5/1049; A61B 19/54; A61N 2005/1058; A61N 2005/1059; A61N 2005/1087; A61B 2019/5462
USPC 600/1

(57) ABSTRACT

Systems and methods of tracking location of an internal bodily structure of a patient in a radiation treatment room, including a fiducial marker having a unique center point, an offset structure detachably connected to the fiducial marker, the offset structure having unique three dimensional offset coordinates relative to the center point, a means for detachably mounting the offset structure to the patient, an imaging unit to measure location information of the offset structure relative to a target internal bodily structure of the patient, and a detection unit to detect location information of the offset structure and to calculate an offset distance between the target internal bodily structure and the center point.

### CROSS-REFERENCE TO RELATED APPLICATIONS

This application claims the benefit of U.S. Provisional Patent Application Ser. No. 61/879,873, filed on Sep. 19, 2013, the disclosure of which is incorporated herein in its entirety by reference.

### FIELD OF INVENTION

The present general inventive concept relates to systems and methods of tracking the location of an internal bodily structure of a patient using external markers.

### BACKGROUND

In some medical applications such as proton therapy (PT), it is desirable to track the location of target areas in the human body. For regions of the human anatomy that move, for example due to breathing or heartbeat, it is important to take such motions into consideration, when computing the effect of the motion on the treatment plan being generated. PT is a cancer treatment technology that uses high energy protons to penetrate a patient's body and deposit energy into treatment volumes such as cancerous tumors. The charged protons may be generated in a particle accelerator, commonly referred to as a cyclotron and/or a synchrotron, and directed to the patient in the form of a beamline using a series of magnets that guide and shape the particle beamline such that the particles penetrate the patient's body at a selected location and are deposited at the site of the treatment volume. Particle therapy leverages the Bragg Peak property of charged particles such that the majority of the energy is deposited within the last few millimeters of travel along the beamline—at a point commonly referred to as the isocenter, as opposed to conventional, intensity modulated radiation therapy (i.e., photons) in which the majority of energy is deposited in the first few millimeters of travel, and the radiation can pass beyond the target region, thereby undesirably damaging healthy tissue.

Fiducial markers have been used in the past, in order to track target regions of the anatomy. Fiducials-based tracking can be difficult for a patient, for a number of reasons. For example, high accuracy tends to be achieved by using bone-implanted fiducial markers, but implantation of fiducials into a patient is generally painful and difficult. Less invasive techniques such as skin-attached markers have been used, but such systems are typically less accurate, especially when the target area is moving, for example during respiration or heart beating of the patient. In some methods that use gating to handle anatomical motion, dynamic tracking may be achieved by establishing a relationship between internally implanted fiducials, and externally placed markers that are tracked in real time. Multiple doses of radiation are often used to track the location of a target area for treatment.

Target positioning through imaging guidance is important for the accurate delivery of radiation treatment. It is challenging to verify that the imaging, localization, and targeting systems are aligned with the true radiation isocenter. Accordingly, systems and methods of tracking internal structures that are less invasive, more accurate, less time consuming, and more effective would be desirable.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates an external fiducial marker configured in accordance with an example embodiment of the present general inventive concept;

FIG. 2 illustrates a proton therapy treatment room during an isocenter set-up phase according to an example embodiment of the present general inventive concept;

FIGS. 3A and 3B illustrate a proton therapy treatment room during patient set-up and operational phases according to an example embodiment of the present general inventive concept; and

FIG. 4 illustrates a proton therapy treatment room configured in accordance with an example embodiment of the present general inventive concept.

### DETAILED DESCRIPTION

Reference will now be made to the example embodiments of the present general inventive concept, examples of which are illustrated in the accompanying drawings and illustrations. The example embodiments are described herein in order to explain the present general inventive concept by referring to the figures.

Various example embodiments of the present general inventive concept provide systems and methods of tracking the location of an internal bodily structure of a patient. These systems and methods may help to provide accurate tumor localization, and may be used to deliver radiation beams to the target tumor with minimal x-ray invasions.

Embodiments of the present general inventive concept provide various tumor localization techniques to precisely determine the location of tumor(s) to help ensure that an effective dose of radiation is delivered to the tumor(s), while sparing healthy, non-cancerous tissue. On-board imaging technologies such as single and stereoscopic x-ray imaging, kilovoltage and megavoltage CT imaging, implantable fiducial markers and transponders, ultrasound imaging, MRI, and others may help to improve the efficacy of proton or other radiation therapy by gathering tumor location information such that a radiation beam may be specifically targeted at the tumor region. Various proton beam-shaping techniques may also be used to help direct radiation precisely at the tumor(s) to be treated, while reducing the radiation exposure to surrounding tissue.

In some embodiments, a Cone Beam Computed Tomography (CBCT) imaging system can be used to deliver 3-d images to permit registration between a 3-d reference image and a 3-d current image, improving precision of patient positioning. The CBCT can also be moved to a specific position and take a flat, digital x-ray image for confirmation images. Diagnostic imaging modalities may be integrated, initially to support academic research advancing proton therapy methods.

Digital x-ray, using an x-ray tube source and flat x-ray panels, are commonly used to image patients. These systems are quick to image and give a relatively low radiation dose to the patient for each image. However, the baseline image used for registration (alignment of the patient images with the planning image) is a 3-D image from the planning CT, so there can be a loss of fidelity when attempting to register 2-D images with the 3-D baseline. Two orthogonal x-rays may be taken to register in two different views. Two x-rays at an oblique angle can be used to create a single, stereoscopic image for registration.

Imaging with orthogonal x-rays gives the patient an additional dose of radiation which is roughly 0.2% of the proton therapy dose.

CBCT can be used to produce a 3-D image with lower soft tissue contrast than the diagnostic CT used for planning. The image from CBCT can be used for the purpose of registration.

Imaging with CBCT gives the patient an additional dose of radiation. For a typical, two-field treatment CBCT delivers in x-ray dose roughly 0.7% of the proton therapy dose, or about three times the dose associated with a pair of planar x-rays.

Diagnostic CT can be used to reproduce the fidelity of the planning CT image. Imaging with DXCT has the disadvantage of higher dose to the patient. At about 12 times the dose associated with planar x-rays, over the treatment period the patient will receive about 3% of the proton therapy dose in additional x-ray dose, a value large enough to warrant inclusion in treatment planning.

In some embodiments, an alternative to imaging the patient with DXCT at every treatment is to use a more conservative imaging approach for daily use, and image the patient infrequently on DXCT, timing to be set by the interval for re-planning treatment. This can be accomplished by including two imagers on the gantry or by adding the DXCT to the treatment room as an accessory, using the Patient Positioning Subsystem to present the patient to the DXCT. CT-on-rails can work with a couch which does not move. Another option is to do infrequent DXCT imaging in another location. However, if the CT is done in conjunction with PET scanning to image the location of delivered radiation (see Section 0 below0 below), imaging while the patient is still on the PPS may be desirable.

Magnetic Resonance Imaging sends magnetic fields into the patient to reconstruct internal structures. MRI allows for patient tracking during setup without radiation exposure. Additionally, it can be used in conjunction with PET for doseless range verification.

A Vision System can be implemented that uses a camera to locate the patient via pixel coordinates from a camera. If the registered pixels change, it can be determined that the patient has moved. This external structure can be overlaid with the internal x-ray image to perform patient motion tracking.

Infrared Tracking (IR) can be used to track external structures of the patient or an external fiducial. As the IR beam bounces from the external structure back to the sensor, the time can then be used to create a 3D location of the structure. If the patient external structure moves (or the fiducial on the patient) then the patient positioning system can adjust accordingly. This patient tracking determines patient movement without additional radiation exposure.

Inertial motion units (IMU) sensors can include, among other things, accelerometers, magnetometers, and gyroscopes that measure changes in the rotational forces being applied to the sensor. The vectors obtained can then be used for reverse kinematics to determine the translational changes to the sensor. Here, the sensors can be used to detect and quantify patient motion without additional radiation exposure.

Both Ultrasound and Microwave imaging technologies provide information about the patient's internal structures without exposing the patient to radiation.

For example, ultrasound imaging can use high frequency waves (e.g., 1-7 MHz) to detect density differences between hard and soft tissues. An Ultrasound piezo transducer coupled with electromagnetic (EM) tracking, vision system tracking, interferometer tracking, or equivalent tracking technology can create a 3D reconstruction of the patient's internal tissue. Once the tumor's position relative to hard tissue has been located via x-ray, CBCT, or DXCT image then the hard tissue can be located with the Ultrasound transducer without additional radiation.

Microwave imaging uses higher frequency waves (1-5 GHz) to detect dielectric differences between various soft tissues. In cases where Ultrasound cannot definitively discern between soft tissue and a tumor, Microwaves can due to the higher water content of a tumor compared to the surrounding tissue. This higher water content raises the dielectric constant such that the tumor can be located within the patient. Similar to Ultrasound, the Microwave transducer can be tracked without radiation.

In addition to standard transducers, embodiments of the present general inventive concept can implement treatment specific probes. For instance, Prostate treatments can involve insertion of a saline filled rectal balloon. A small, EM tracked Microwave probe can be inserted inside this balloon to locate the tumor in real time. Such techniques can be applied with Ultrasound except the device may be tracking internal hard tissue instead of the tumor itself.

For some general applications, a 3D tracked Ultrasound probe can be placed externally on the patient near the tumor. Once an x-ray image has been collected, this probe (or array of probes) can track the location of the hard tissue and provide tracking of the tumor without additional radiation exposure.

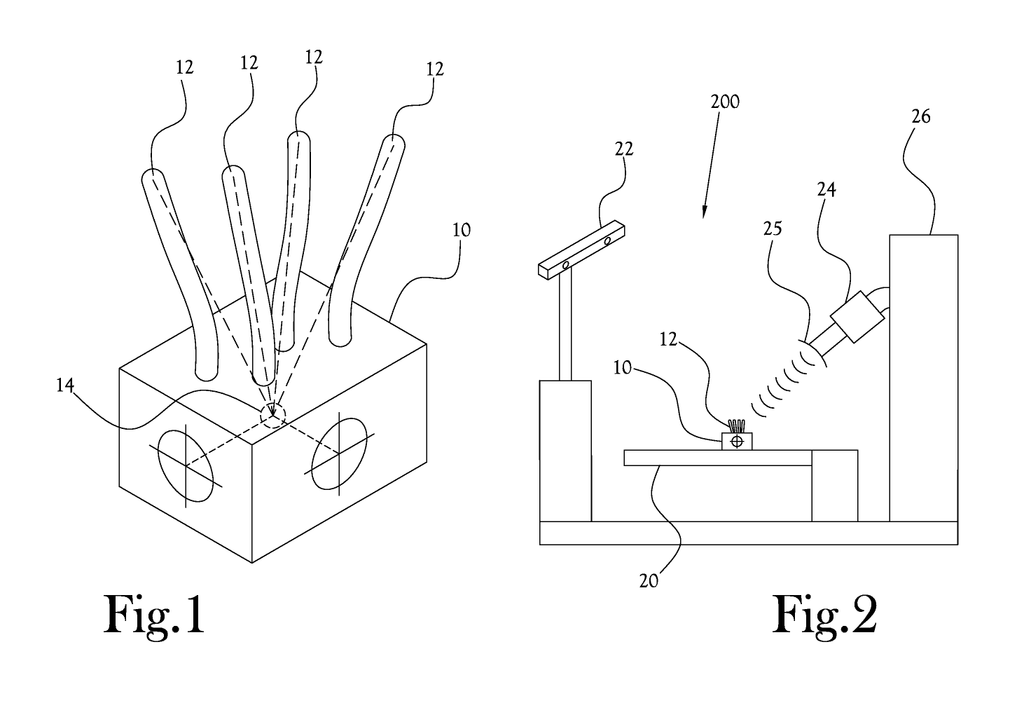

FIG. 1 illustrates an external fiducial marker configured in accordance with an example embodiment of the present general inventive concept. Although the present general inventive concept contemplates the use of any 3-d surface of the patient to track the location of tumors, some embodiments utilize an external fiducial marker, such as the example fiducial marker illustrated in FIG. 1, to assist the location calculus.

As illustrated in FIG. 1, an example embodiment of the present general inventive concept can include an external fiducial marker 10 configured to provide an accurate and efficient means of determining radiation isocenter 14 coincidence with the isocenters of image guided systems. The fiducial marker 10 can include an offset structure which in this embodiment comprises a plurality of detachable fingers 12 detachably coupled to the fiducial marker via a detachment member. The detachment member can take various forms chosen with sound engineering judgment, for example a mechanical and/or magnetic interlocking structure to precisely locate and secure the offset structure 12 and marker 10 in an appropriate orientation one to the other. The fingers 12 are configured in shape and size to provide a unique 3-d offset reference to the center point 14 of the fiducial 10 which can be mapped to the isocenter of the proton delivery system. A variety of other shapes and sizes could be chosen using sound engineering judgment in addition to a ‘finger’ configuration as illustrated herein to represent and determine a true radiation isocenter corresponding to isocenter 14 of the marker 10.

As illustrated in FIG. 2, the fiducial marker 10 can be placed on the patient bed 20 within a proton therapy treatment room 200 during an isocenter set-up phase. The patient bed can include a mounting structure or receptacle to receive the fiducial marker 10 to relate the isocenter to a predetermined location of the patient bed. The treatment room can include a gantry 26 to rotate a proton beam nozzle 24 about a patient to be positioned on the patient bed 20. The example treatment room environment of FIG. 2 includes a detection unit 25, such as, but not limited to, an infrared detector 25, positioned on the proton beam nozzle 24, to detect relative position information of the marker 10 and fingers 12 relative to the isocenter of the marker. An optional camera unit 22 may also be provided in the treatment room to detect location information of components.

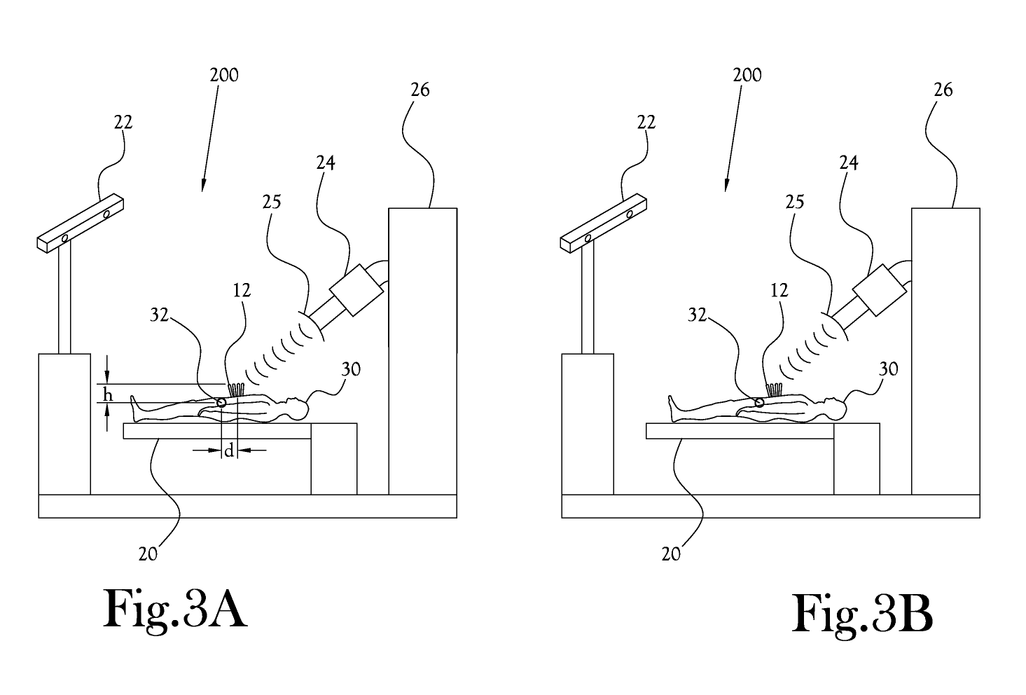

As illustrated in FIGS. 3A and 3B, once the radiation isocenter has been established by detecting the fiducial marker 10, a patient 30 can be located on the bed 20, and the offset structure (e.g., detachable fingers) 12 can be placed on the patient. A variety of means can be provided to locate and secure the detachable fingers 12 to a desired location of the patient. Non-limiting examples include a mounting belt having an electro/mechanical interlocking device to receive and orient the offset structure as desired. A variety of other means for placing the offset structure 12 on the patient could be implemented using sound engineering judgment without departing from the broader scope of the present general inventive concept.

Once the offset structure is mounted to the patient, an x-ray or other image of the patient can be taken to determine location information of a tumor 32 relative to the offset structure 12. The detector unit can include a processor having a calculation module comprising various electronic components, switches and/or solid state modules configured to compare and manipulate the location information of the tumor 32 and offset structure 12 so as to determine three dimensional coordinates of the tumor and offset structure 12 in order to determine offset coordinates (e.g., h, d, w) between the tumor and offset structure, enabling an operator or robotic machine to move the patient bed and/or nozzle an appropriate amount corresponding to the offset coordinates h, d, w, such that the isocenter of the tumor 32 can be aligned with the radiation isocenter 14 (see, e.g, FIG. 1) of the proton therapy system based on the location of the offset structure 12 relative to the isocenter of the proton delivery system. It is noted that offset coordinates ‘h’ and ‘d’ are shown in the 2-dimensional rendering of FIG. 3A, but it is understood that a third dimension ‘w’ (in and out of page) could also be provided to provide true 3-dimensional offset coordinates between tumor 32 and offset structure 12, relative to the isocenter 14.

Since the detection unit 25 (e.g., infrared detector) is located on the proton beam nozzle 24, it is possible to measure the air gap between the patient and the nozzle 24, without the necessity of having a treatment assistant enter the treatment room to check and verify the air gap.

Moreover, embodiments of the present general inventive concept enable gating patterns to be obtained by a series of CT's (e.g., fluoroscopy), which can be compared to and/or predicted from a pattern of movements of the external fiducial marker (or other 3-d patient surface) during patient respiration or other anatomical movements.

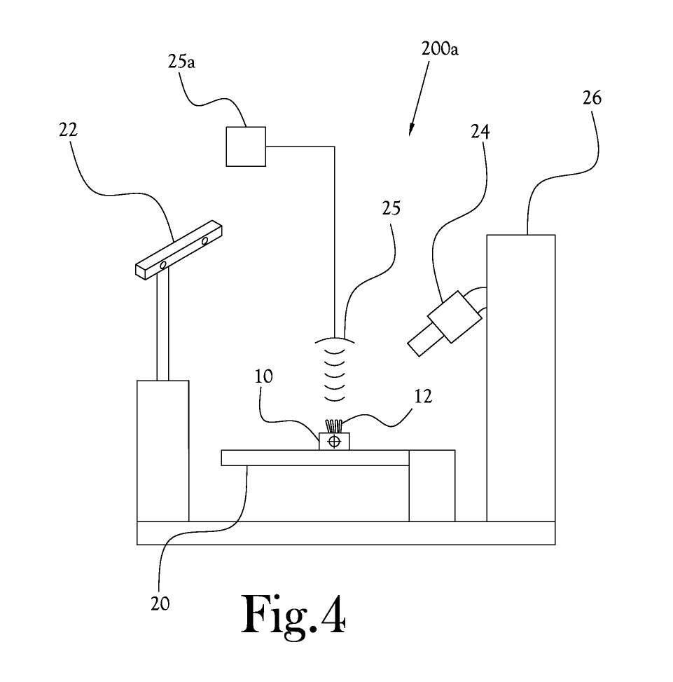

FIG. 4 illustrates a proton therapy treatment room 200a configured in accordance with another example embodiment of the present general inventive concept. As illustrated in FIG. 4, the detection unit 25 can be floating in space to enable location flexibility of the detection unit 25. For example, in some embodiments, the detection unit can be an infrared detector 25 to determine location information of the fiducial marker 10, 12, and the camera unit 22 can be used to capture location information of the infrared detector. Accordingly, once the x-ray unit captures the location of the tumor relative to the marker 12, it is possible to calculate the relative location of the fiducial marker to the tumor, thus knowing where everything is.

It is noted that the simplified diagrams and drawings do not illustrate all the various connections and assemblies of the various components, however, those skilled in the art will understand how to implement such connections and assemblies, based on the illustrated components, figures, and descriptions provided herein, using sound engineering judgment.

Numerous variations, modifications, and additional embodiments are possible, and accordingly, all such variations, modifications, and embodiments are to be regarded as being within the spirit and scope of the present general inventive concept. For example, ultrasound, microwave, or other known or later developed technology could be used instead of IR (infrared) to achieve the same or similar results. Microwave transducers could be placed on the patient's body to obtain relative location information to the tumor using microwaves.

In addition, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. A system to track location of an internal bodily structure of a patient in a radiation treatment room, comprising:
a fiducial marker having a unique center point;
an offset structure detachably connected to the fiducial marker, the offset structure having unique three dimensional offset coordinates relative to the center point;
a means for detachably mounting the offset structure to the patient;
an imaging unit to measure location information of the offset structure relative to a target internal bodily structure of the patient; and
a detection unit to detect location information of the offset structure and to calculate an offset distance between the target internal bodily structure and the center point.

2. The system of claim 1, wherein the radiation treatment room is a proton therapy treatment room, and the detection unit is mounted to a proton beam nozzle of the proton therapy treatment room such that the detection unit detects an air gap between the proton beam nozzle and the patient.

3. The system of claim 1, further comprising a camera unit to detect location information of the detection unit relative to the offset structure.

4. The system of claim 1, wherein the detection unit is one of an infrared detector, ultrasound detector, camera unit, or microwave detector.

5. The system of claim 1, further comprising means for obtaining a gating pattern during movements of the patient and comparing the gating pattern to corresponding movements of the offset structure.

6. A method of tracking location of an internal bodily structure of a patient in a radiation treatment room, comprising:
providing a fiducial marker having a unique center point;
mounting an offset structure to the fiducial marker, the offset structure having unique three dimensional offset coordinates relative to the center point;
coordinating the unique center point of the fiducial marker with a radiation isocenter of the treatment room;
mounting the offset structure to a patient;
measuring location information of the offset structure relative to a target internal bodily structure of the patient; and
measuring location information of the offset structure; and
calculating an offset distance between the target internal bodily structure and the center point based on the location information of the offset structure.

7. The method of claim 6, wherein the radiation treatment room is a proton therapy treatment room, the method further comprising:
mounting the detection unit to a proton beam nozzle of the proton therapy treatment room; and
detecting an air gap between the proton beam nozzle and the patient using the detection unit.

8. The method of claim 6, further comprising detecting location information of the detection unit relative to the offset structure.

9. A system to track location of an internal bodily structure of a patient in a radiation treatment room, comprising:
an imaging unit to measure offset coordinates between an external 3-d structure of the patient and an internal bodily structure of the patient relative to a radiation isocenter of the treatment room; and
a detection unit to detect location information of the 3-d structure and to calculate an offset distance between the target internal bodily structure and the isocenter using the location information of the 3-d structure.

10. A method of tracking location of an internal bodily structure of a patient in a radiation treatment room, comprising:
measuring offset coordinates between an external 3-d structure of the patient and an internal bodily structure of the patient relative to a radiation isocenter of the treatment room;
detecting location information of the 3-d structure; and
calculating an offset distance between the target internal bodily structure and the isocenter using the location information of the 3-d structure.
